(19) United States
(12) Patent Application Publication
(10) Pub. No.: US 2026/0157723 A1
(43) Pub. Date: Jun. 11, 2026

(54) DETECTION, PREDICTION, AND ANALYSIS OF BOWEL WALLS

(71) Applicant: Motilent Limited, London (GB)

(72) Inventors: Alexander Menys, London (GB); Danny Raj Ramasawmy, Cambridge (GB)

(21) Appl. No.: 19/365,486

(22) Filed: Oct. 22, 2025

Related U.S. Application Data
(60) Provisional application No. 63/710,161, filed on Oct. 22, 2024.

(51) Int. Cl. A61B 8/12; A61B 8/00; A61B 8/08

(52) U.S. Cl. CPC A61B 8/12; A61B 8/085; A61B 8/465; A61B 8/5223

(57) ABSTRACT

Systems and methods for detection, prediction, and analysis of bowel walls are discussed. A method for detecting a bowel of a patient includes: performing an intestinal ultrasound on a the patient; determining, using an artificial intelligence/machine learning (AI/ML) model, whether a bowel is detected in data corresponding to of the intestinal ultrasound; and categorizing the data corresponding to the intestinal ultrasound based on the determination. Corresponding computer systems for implementing these mechanisms (and for storing and/or implementing instructions for the same) are also discussed.

### CROSS-REFERENCE TO RELATED APPLICATION

This application claims the benefit, under 35 U.S.C. § 119(e), of U.S. provisional application No. 63/710,161, entitled “Detection and Prediction of Bowel Walls” and filed on Oct. 22, 2024, which is hereby incorporated in its entirety by reference herein.

### TECHNICAL FIELD

The present disclosure relates generally to systems and methods for detecting bowel walls using ultrasound and performing processing of corresponding ultrasound data.

### BACKGROUND

Intestinal Ultrasound (IUS) is an important emerging tool to evaluate and treat disease activity in Crohn's Disease and Ulcerative Colitis as well as other pathologies (e.g., Scleroderma). IUS provides a low cost and widely accessible technology to assess bowel wall thickness, motility, presence of fat and changes in blood flow that help in the assessment of disease state.

In some cases, IUS may be used in research (e.g., academic and clinical trials) as well as in clinical implementations. However, there may be a long training and ramp up time to become proficient with IUS both to spot pathology as well as follow up on the pathology. In clinical trials, it is common for sites to miss diseases and/or to upload excessive amounts of data to avoid leaving out important frames in the central reading process. Additionally, a large amount of time may be taken to read through this data to spot key frames for disease detection. Further, identifying disease is challenging outside of severe cases in trials and clinical practice alike and as such a large amount of skill is needed to re-identify the same location and confidently call for a treatment response.

IUS technology is becoming cheaper and more available. Low cost handheld options provide avenues for use in clinics where, for example, assistance may be needed in the event that less-skilled, non-expert operators are given access. For example, in inflammatory bowel disease (IBD) based implementations, IBD nurses and physicians'assistants not trained in IUS technologies may perform IUS alongside their other clinical duties, even though not trained for IUS. Gastroenterologists may soon utilize IUS technologies, however IUS is secondary to their main clinical procedures (e.g., endoscopy) and they may not have time for comprehensive training corresponding to the IUS. Additionally, a future cohort of patients and careers who may perform IUS at home to avoid clinical visits may not have time or the ability to undergo complex and time consuming training that comes along with utilizing IUS technology.

### SUMMARY

A method for detecting a bowel of a patient is disclosed. In one embodiment, the method includes performing an intestinal ultrasound on a patient; determining, using an artificial intelligence/machine learning (AI/ML) model, whether a bowel is detected in data corresponding to the intestinal ultrasound; and categorizing the data corresponding to the intestinal ultrasound based on the determination.

Optionally, in some embodiments, the method further includes scoring a frame of the data corresponding to the intestinal ultrasound.

Optionally, in some embodiments, scoring the frame of the data corresponding to the intestinal ultrasound comprises giving the frame a score of: a first value (e.g., 0) when no bowel is detected in the frame; a second value (e.g., 1) when some bowel is detected in the frame; a third value (e.g., 2) when a bowel is detected in the frame; or a third value (e.g., 3) when a bowel is detected in subsequent frames.

Optionally, in some embodiments, scoring a frame of the data corresponding to the intestinal ultrasound comprises giving the frame a confidence score corresponding to a detection of the bowel.

Optionally, in some embodiments, the method further includes aggregating the scoring of the frame of the data corresponding to the intestinal ultrasound. Optionally, in some such embodiments, aggregating the scoring is based on a severity score. Optionally, in some such embodiments, aggregating the scoring is based on a time-frame score.

Optionally, in some embodiments, the method further includes determining whether the bowel detected in the data corresponding to the intestinal ultrasound comprises a disease.

Optionally, in some embodiments, the method further includes presenting, to a user, the data corresponding to the intestinal ultrasound on a graphical user interface on a display.

Optionally, in some embodiments, the method further includes providing an indication to a user whether the bowel is detected in the data corresponding to the intestinal ultrasound.

### BRIEF DESCRIPTION OF THE DRAWINGS

FIG. 1 illustrates a simplified schematic of a system for detecting a bowel, according to embodiments herein.

FIG. 2 illustrates an example of a scoring IUS frames corresponding to a performed IUS, according to embodiments herein.

FIG. 3A, FIG. 3B, FIG. 3C, and FIG. 3D illustrate imaging (FIG. 3A) and related plotted image data (FIG. 3B, FIG. 3C, and FIG. 3D) corresponding to a performed IUS, according to embodiments herein.

FIG. 4A, FIG. 4B, FIG. 4C, and FIG. 4D illustrate imaging (FIG. 4A) and corresponding plotted image data (FIG. 4B, FIG. 4C, and FIG. 4D) corresponding to a performed IUS, according to embodiments herein.

FIG. 5 illustrates a graphical user interface showing an ultrasound volume with thickened bowel and a 0 to 3 scoring, according to embodiments herein.

FIG. 6 illustrates a simplified block diagram of components of a computing system of the system for predicting and mitigating the effects of environmental conditions of FIG. 1, according to embodiments herein.

### DETAILED DESCRIPTION

Embodiments herein provide a supportive methodology to simplify acquisition and support widespread operationalization of IUS technology, without the need of training.

In some embodiments using artificial intelligence/machine learning (AI/ML), a bowel may be identified in IUS frames received from performing IUS. Subsequently, the frames may be labeled with a sensitivity and a specificity. Then, potential disease activity may be graded within the frames. The results are presented visually in a number of ways for onward processing and presentation to the user.

In some embodiments, the AI/ML tool (which may be referred to herein as a “HitRATE AI/ML model”) may automatically grade IUS video frames on a scale (e.g., a 0-3 scale) for the presence of bowel wall (in cases of a small and a large bowel). A first value (e.g., 0) may correspond to no bowel wall being detected. A second value (e.g., 1) may correspond to a partial bowel wall being detected. A third value (e.g., 2) may correspond to a clear bowel wall being detected. A third value (e.g., 3) may correspond to a clear bowel wall with category 2 data on either side (in time) being detected (i.e., subsequent frames also have a bowel detected).

In some cases, the HitRATE AI/ML model may be run either during the procedure or at the point of ultrasound upload to a contract research organization or clinic to provide a first pass check of viable data entering the study. Together with a protocol that introduces systemized data collection, the HitRATE AI/ML model may reduce missed loops or erroneous data collection.

In some embodiments, the image values and resulting values from the HitRATE AI/ML model may be plotted into a visual plot to assist with automated extraction of key frames and/or to provide real time feedback to a user to help the user understand when the user is collecting the correct data (or to understand that the user is not collecting correct data). For example, a graphical user interface may be introduced showing, to the user, an ultrasound volume with thickened bowel and the 0 to 3 scoring discussed herein as a plot.

In some embodiments, a clinical severity of a disease might be profiled in order to conduct a component of or a complete disease activity index that facilitates clinical evaluation and/or data collection. For example, the HitRATE AI/ML model may profile a disease detected in the bowel and provide data corresponding to the disease to facilitate clinical evaluation.

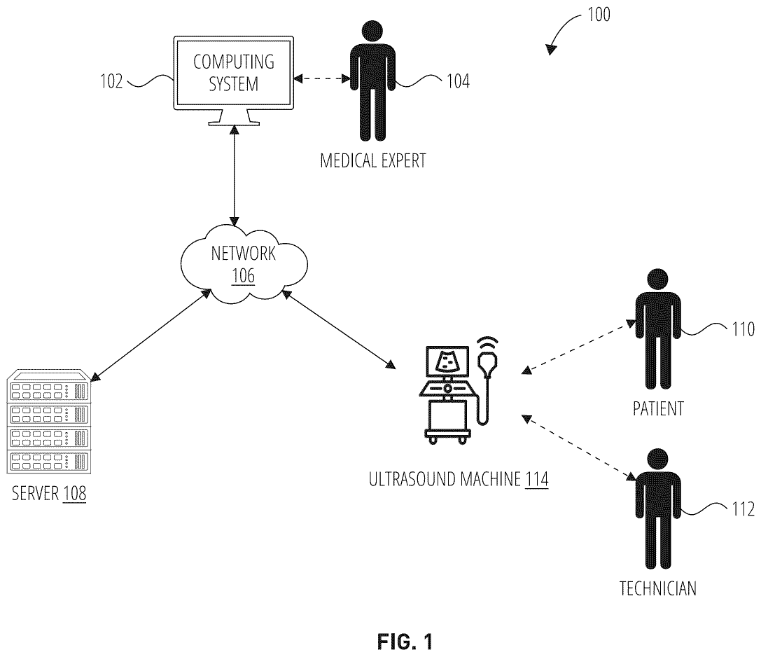

Turning to the figures, FIG. 1 illustrates a simplified schematic of a system 100 for detecting a bowel, according to embodiments herein.

The system 100, which may be used to perform or assist in performing any of the embodiments discussed herein, includes a server 108, a network 106, an ultrasound machine 114, which a patient 110 and/or a technician 112 may interact with, and a computing system 102, which a medical expert 104 may interact with.

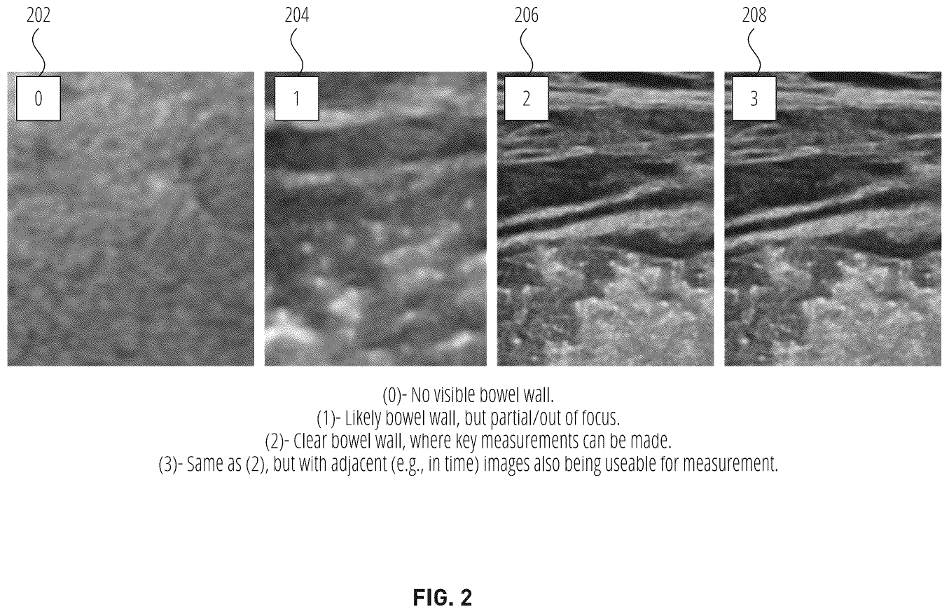

FIG. 2 illustrates an example of scoring IUS frames corresponding to a performance of an IUS, according to embodiments herein.

In some examples, when there is no visible bowl wall, the score given to the frame is 0 202. In some examples, when there likely is a bowel wall but it is partial/out of focus, the score given to the frame is 1 204. In some examples, when there is a clear bowel wall where key measurements may be made, the score given to the frame is 2 206. In some examples, the criteria for a score of 3 208 is the same as a score of 2 206, but a score of 3 208 given to a frame means that adjacent, equally clear images that are suitable for purposes of measurement are available (e.g., in other, adjacent, image frames that precede and/or follow this frame in time).

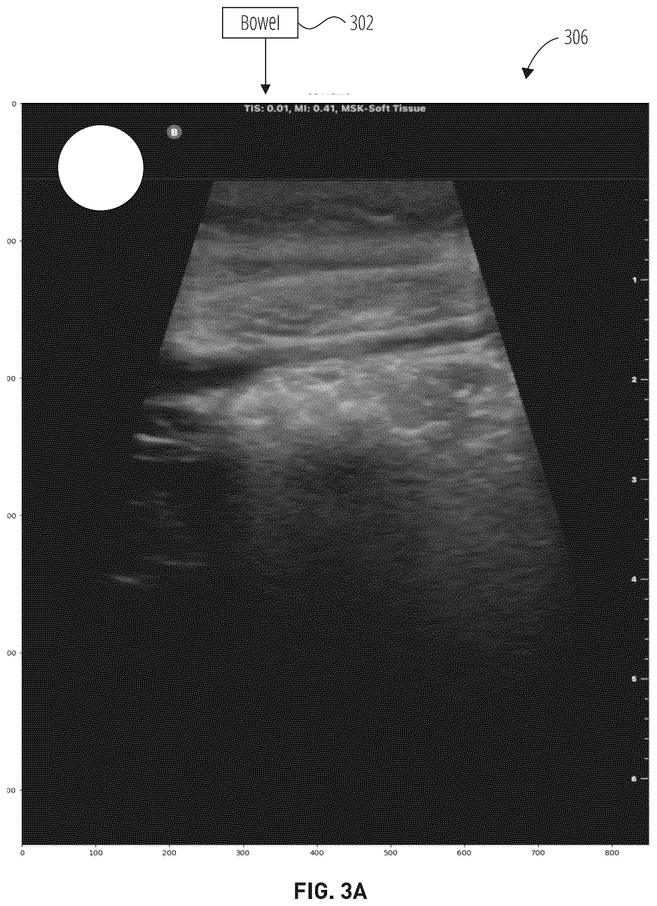

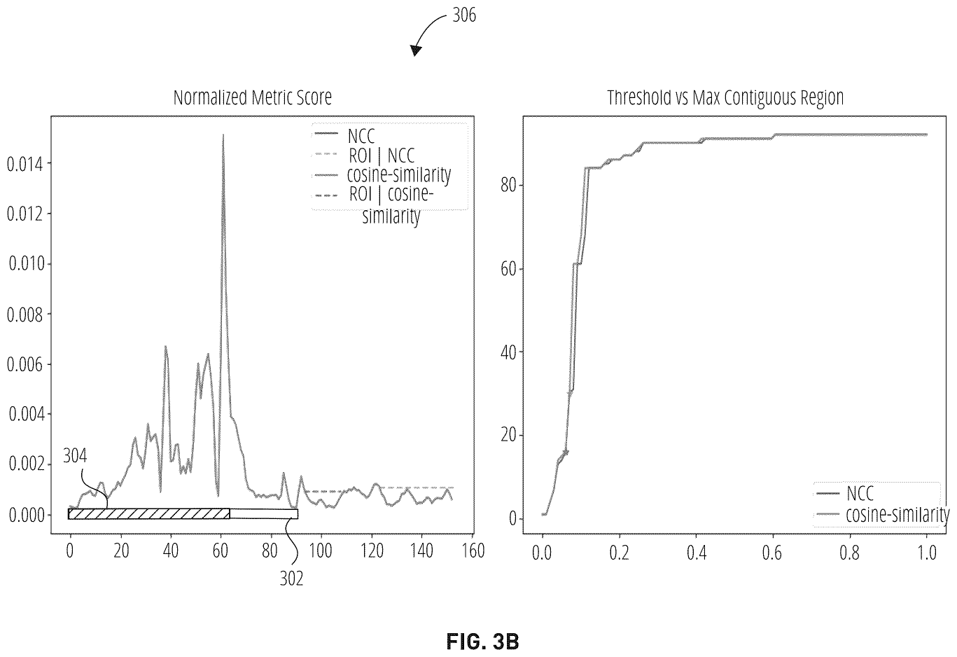

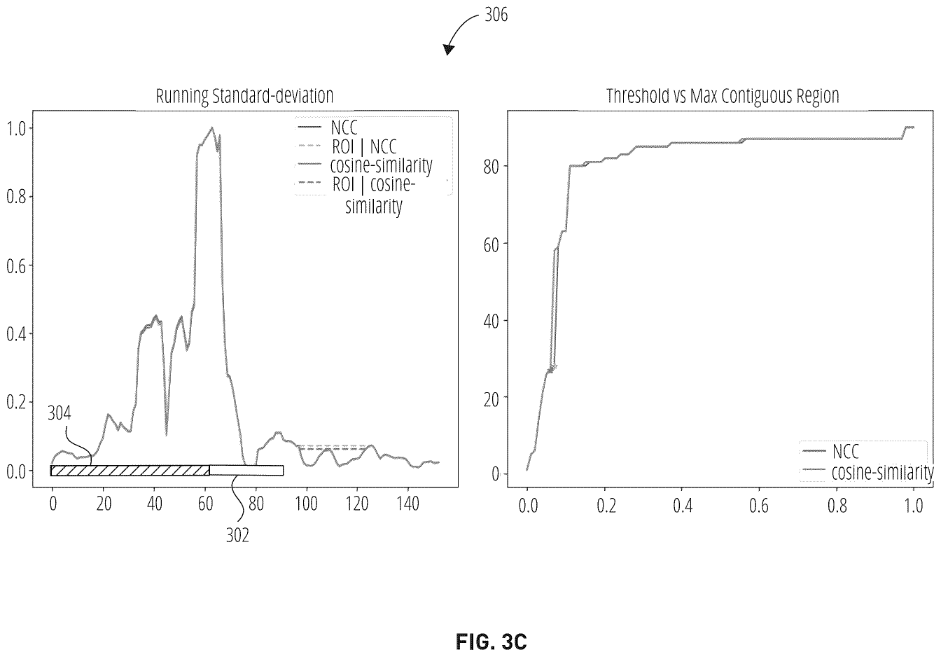

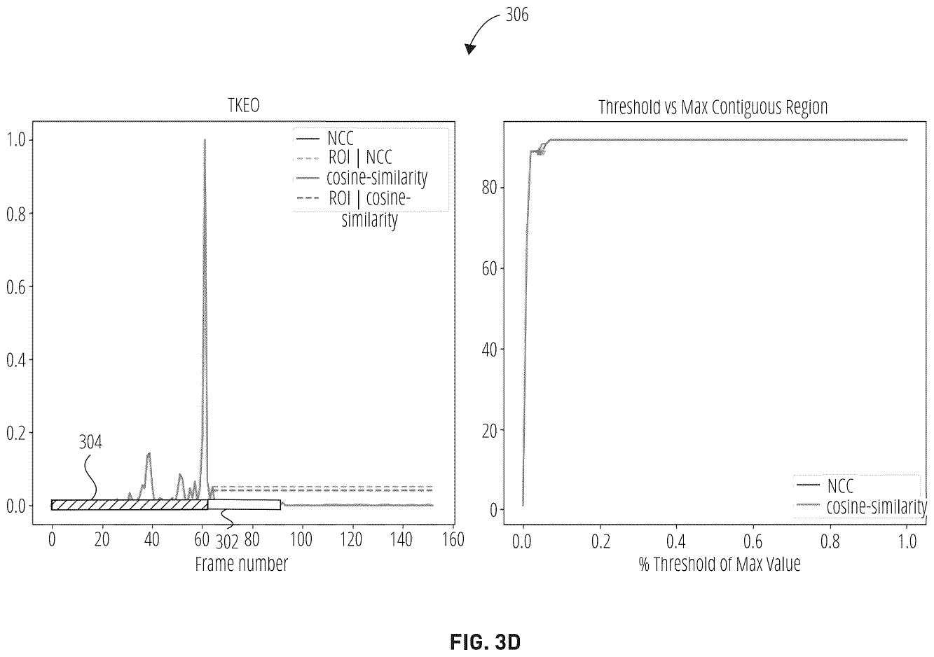

FIG. 3A, FIG. 3B, FIG. 3C, and FIG. 3D illustrate imaging (FIG. 3A) and related plotted image data (FIG. 3B, FIG. 3C, and FIG. 3D) corresponding to a performed IUS, according to embodiments herein.

In some examples, when a bowel is detected 306 when performing an IUS, image data corresponding to the IUS may be plotted showing when the bowel is detected 302 transitioning from when the bowel is not detected 304.

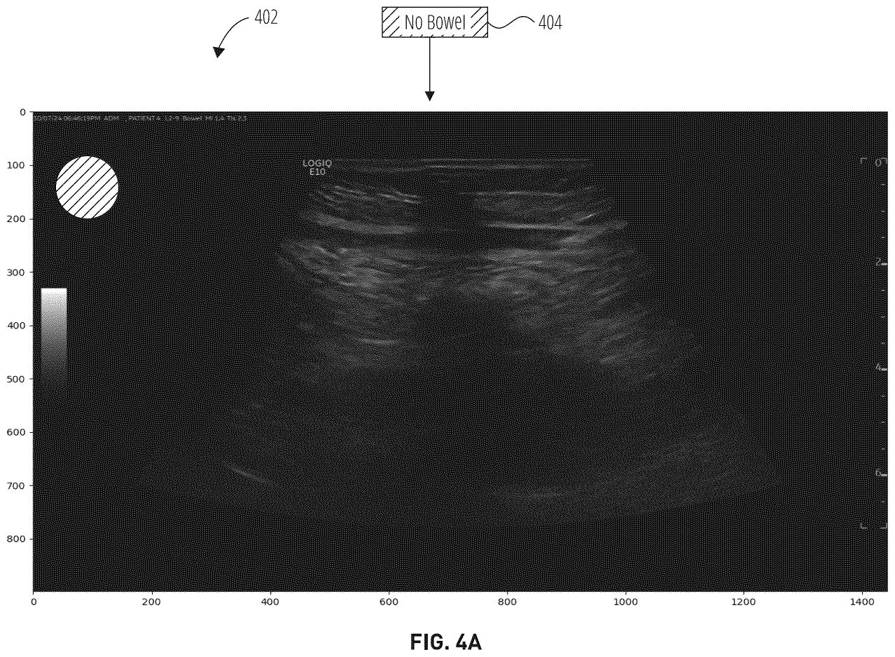

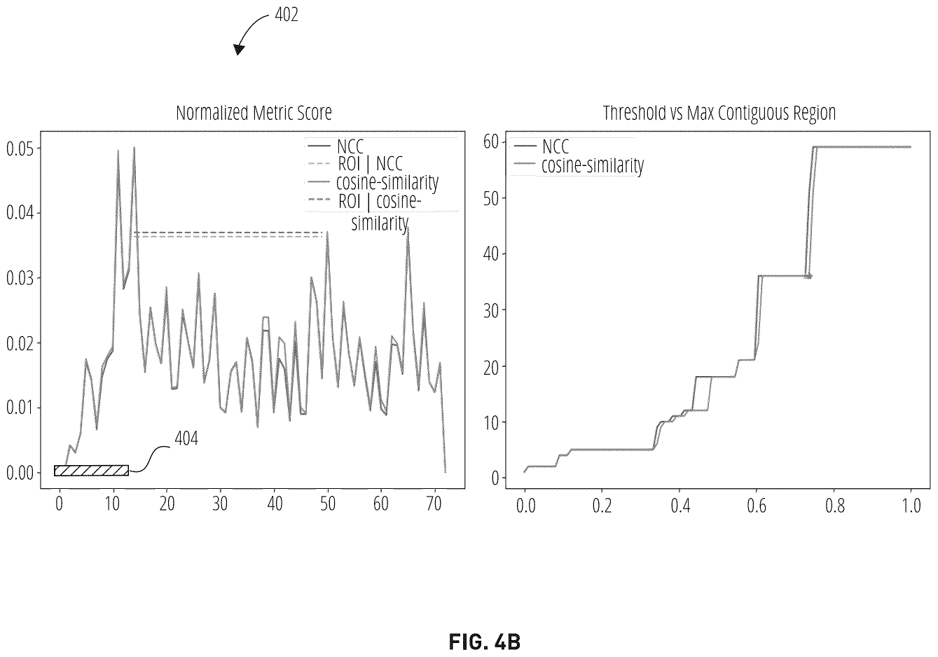

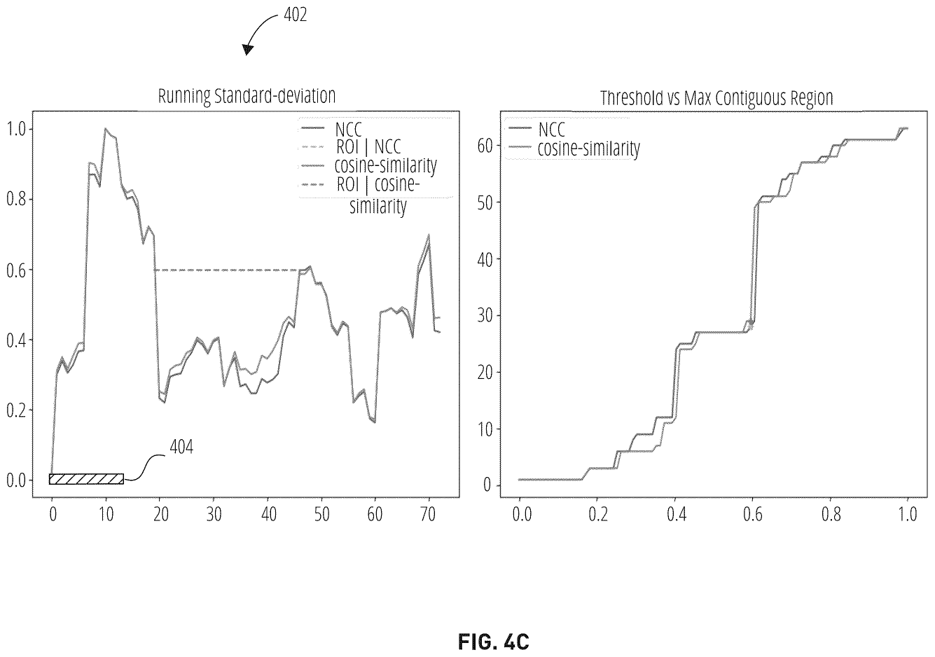

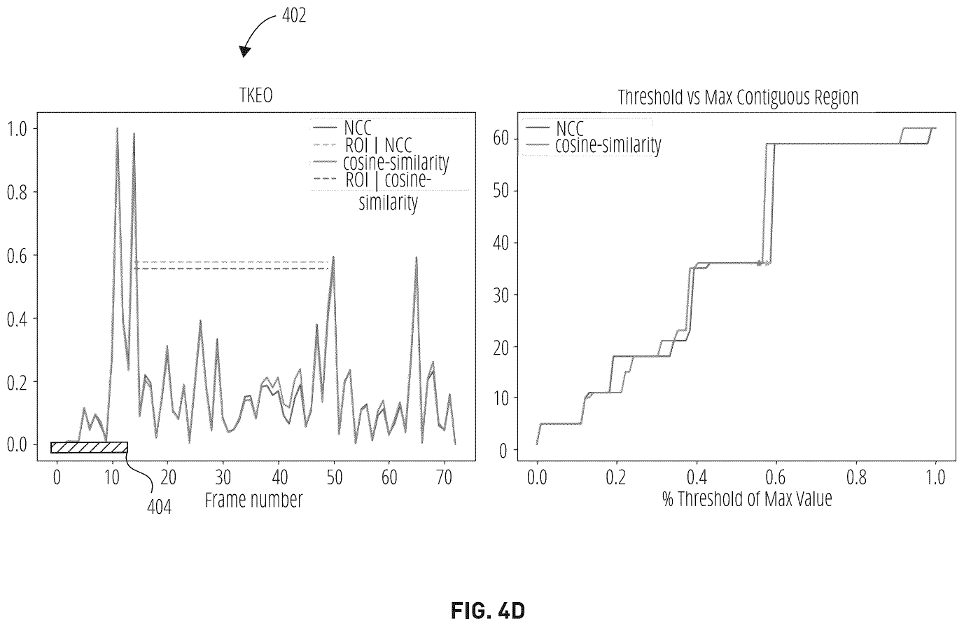

FIG. 4A, FIG. 4B, FIG. 4C, and FIG. 4D illustrate imaging (FIG. 4A) and corresponding plotted image data (FIG. 4B, FIG. 4C, and FIG. 4D) corresponding to a performed IUS, according to embodiments herein.

Similarly, when a bowel is not detected 402 when performing an IUS, image data corresponding to the IUS may be plotted showing that the bowel is not detected 404.

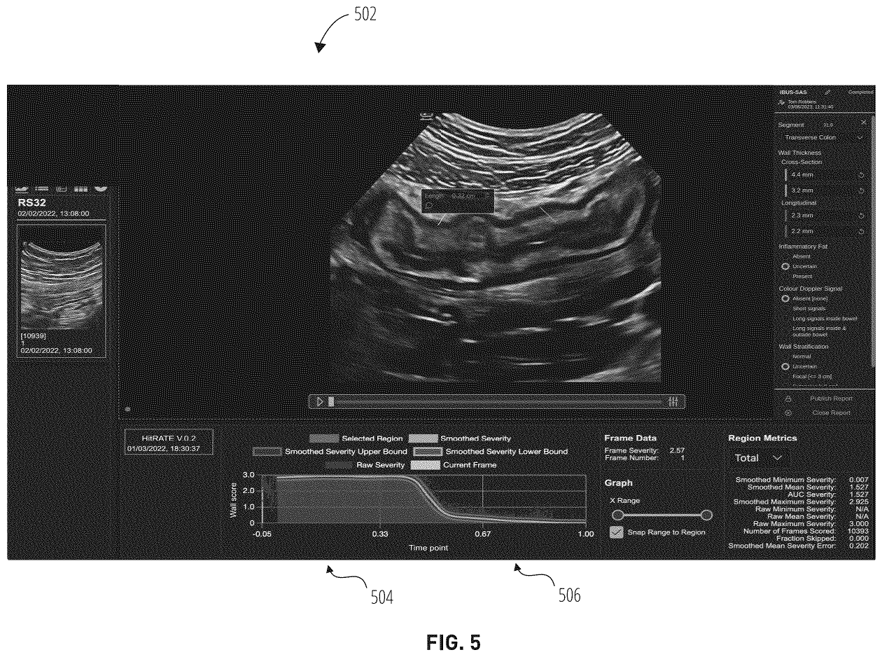

FIG. 5 illustrates a graphical user interface 502 showing an ultrasound volume with thickened bowel and a 0 to 3 scoring, according to embodiments herein.

In some cases, the graphical user interface 502 may be used to present the ultrasound volume of the IUS and a 0 to 3 scoring for the IUS frame. The example graphical user interface 502 illustrates that high quality data occupies a first portion 504 of the clip corresponding to the IUS data (i.e., a score of 3) and that low quality data (i.e., non-bowel corresponding to a score of 0) occupies a latter portion 506. Additionally, an international bowel ultrasound segmental activity score (IBUS-SAS) is present in the graphical user interface 502 which has been partially calculated via extraction from HitRATE AI/ML model.

### Additional Example Embodiments

Certain embodiments disclosed herein ingest a volume of intestinal ultrasound data.

Certain embodiments disclosed herein identify a bowel across a range of disease states (e.g., Crohn's Disease and Ulcerative Colitis) as well as normal bowel within a single frame with a confidence score.

Certain embodiments disclosed herein apply thresholds to the confidence score to categorize data into, for example, 0 no bowel present, 1 some bowel present and 2 high certainty of bowel present.

Certain embodiments disclosed herein grade bowel as diseased or not diseased or on a disease spectrum.

Certain embodiments disclosed herein aggregate and visualized bowel scoring (e.g., on a time-frame score scale).

Certain embodiments disclosed herein aggregate data by severity.

Certain embodiments disclosed herein bookmark and present key frames to assist in reading and interpretation.

Certain embodiments disclosed herein provide real-time feedback on frames containing a bowel or a disease.

Certain embodiments disclosed herein convert confidence into non-visual information (e.g., sound) or symbols to guide non-clinical use (e.g., patients performing self-scanning).

Certain embodiments disclosed herein convert bowel scores into indices.

Certain embodiments disclosed herein present data as part of multi-modality or multi-measurement within modality index.

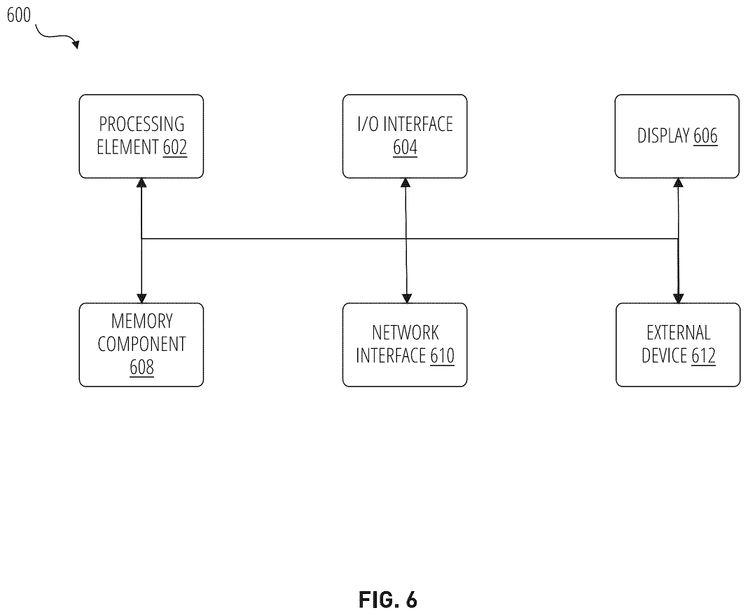

FIG. 6 is a simplified block diagram of components of a computing system 600 of the system of FIG. 1 according to embodiments herein. For example, the processing element 602 and the memory component 608 may be located at one or in several computing systems 600. This disclosure contemplates any suitable number of such computing systems 600. For example, the server may be a desktop computing system, a mainframe, a blade, a mesh of computing systems 600, a laptop or notebook computing system 600, a tablet computing system 600, an embedded computing system 600, a system-on-chip, a single-board computing system 600, or a combination of two or more of these. Where appropriate, a computing system 600 may include one or more computing systems 600; be unitary or distributed; span multiple locations; span multiple machines; span multiple data centers; or reside in a cloud, which may include one or more cloud components in one or more networks. A computing system 600 may include one or more processing elements 602, an input/output I/O interface 604, one or more external devices 612, one or more memory components 608, and a network interface 610. Each of the various components may be in communication with one another through one or more buses or communication networks, such as wired or wireless networks. The components in FIG. 6 are exemplary only. In various examples, the computing system 600 may include additional components and/or functionality not shown in FIG. 6.

The processing element 602 may be any type of electronic device capable of processing, receiving, and/or transmitting instructions. For example, the processing element 602 may be a central processing unit, microprocessor, processor, or microcontroller. Additionally, it should be noted that some components of the computing system 600 may be controlled by a first processing element 602 and other components may be controlled by a second processing element 602, where the first and second processing elements may or may not be in communication with each other.

The I/O interface 604 allows a user to enter data in to computing system 600, as well as provides an input/output for the computing system 600 to communicate with other devices or services. The I/O interface 604 can include one or more input buttons, touch pads, touch screens, and so on.

The external device 612 are one or more devices that can be used to provide various inputs to the computing systems 600, e.g., mouse, microphone, keyboard, trackpad, sensing element (e.g., a thermistor, humidity sensor, light detector, etc. The external devices 612 may be local or remote and may vary as desired. In some examples, the external devices 612 may also include one or more additional sensors.

The memory components 608 are used by the computing system 600 to store instructions for the processing element 602, as well as store data. The memory components 608 may be, for example, magneto-optical storage, read-only memory, random access memory, erasable programmable memory, flash memory, or a combination of one or more types of memory components.

The network interface 610 provides communication to and from the computing system 600 to other devices. The network interface 610 includes one or more communication protocols, such as, but not limited to Wi-Fi®, Ethernet, Bluetooth®, etc. The network interface 610 may also include one or more hardwired components, such as a Universal Serial Bus (USB) cable, or the like. The configuration of the network interface 610 depends on the types of communication desired and may be modified to communicate via Wi-Fi®, Bluetooth®, etc.

The display 606 provides a visual output for the computing system 600 and may be varied as needed based on the device. The display 606 may be configured to provide visual feedback to a user and may include a liquid crystal display screen, light emitting diode screen, plasma screen, or the like. In some examples, the display 606 may be configured to act as an input element for a user through touch feedback or the like.

Any description of a particular component being part of a particular embodiment, is meant as illustrative only and should not be interpreted as being required to be used with a particular embodiment or requiring other elements as shown in the depicted embodiment.

All relative and directional references (including top, bottom, side, front, rear, and so forth) are given by way of example to aid the reader's understanding of the examples described herein. They should not be read to be requirements or limitations, particularly as to the position, orientation, or use unless specifically set forth in the claims. Connection references (e.g., attached, coupled, connected, joined, and the like) are to be construed broadly and may include intermediate members between a connection of elements and relative movement between elements. As such, connection references do not necessarily infer that two elements are directly connected and in fixed relation to each other, unless specifically set forth in the claims.

The present disclosure teaches by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall there between.

1. A method for detecting a bowel of a patient comprising:
performing an intestinal ultrasound on the patient;
determining, using an artificial intelligence/machine learning (AI/ML) model, whether a bowel is detected in data of the intestinal ultrasound; and
categorizing the data corresponding to the intestinal ultrasound based on the determination.

2. The method of claim 1, further comprising scoring a frame of the data corresponding to the intestinal ultrasound.

3. The method of claim 2, wherein scoring the frame of the data corresponding to the intestinal ultrasound comprises giving the frame a score of one of:
0, when the bowel is not detected in the frame;
1, when the bowel is partially detected in the frame;
2, when the bowel is detected in the frame; and
3, when the bowel is detected in the frame and in one or more adjacent frames to the frame.

4. The method of claim 2, wherein scoring a frame of the data corresponding to the intestinal ultrasound comprises giving the frame a confidence score corresponding to a detection of the bowel.

5. The method of claim 2, further comprising aggregating the scoring of the frame of the data corresponding to the intestinal ultrasound.

6. The method of claim 5, wherein aggregating the scoring is based on a severity score.

7. The method of claim 5, wherein aggregating the scoring is based on a time-frame score.

8. The method of claim 1, further comprising determining whether the bowel detected in the data corresponding to the intestinal ultrasound comprises a disease.

9. The method of claim 1, further comprising presenting, to a user, the data corresponding to the intestinal ultrasound on a graphical user interface on a display.

10. The method of claim 1, further comprising providing an indication to a user of whether the bowel is detected in the data corresponding to the intestinal ultrasound.

11. A computing apparatus comprising:
one or more processors; and
a memory storing instructions that, when executed by the one or more processor, configure the computing apparatus to:
determine, using an artificial intelligence/machine learning (AI/ML) model, whether a bowel is detected in data of an intestinal ultrasound of a patient; and
categorize the data corresponding to the intestinal ultrasound based on the determination.

12. The computing apparatus of claim 11, further comprising scoring a frame of the data corresponding to the intestinal ultrasound.

13. The computing apparatus of claim 12, wherein scoring the frame of the data corresponding to the intestinal ultrasound comprises giving the frame a score of one of:
0, when the bowel is not detected in the frame;
1, when the bowel is partially detected in the frame;
2, when the bowel is detected in the frame; and
3, when the bowel is detected in the frame and in one or more adjacent frames to the frame.

14. The computing apparatus of claim 12, wherein scoring a frame of the data corresponding to the intestinal ultrasound comprises giving the frame a confidence score corresponding to a detection of the bowel.

15. The computing apparatus of claim 12, further comprising aggregating the scoring of the frame of the data corresponding to the intestinal ultrasound.

16. The computing apparatus of claim 15, wherein aggregating the scoring is based on a severity score.

17. The computing apparatus of claim 15, wherein aggregating the scoring is based on a time-frame score.

18. A non-transitory computer-readable storage medium including instructions that, when executed by one or more processors of a computer, cause the computer to:
determine, using an artificial intelligence/machine learning (AI/ML) model, whether a bowel is detected in data of an intestinal ultrasound of a patient; and
categorize the data corresponding to the intestinal ultrasound based on the determination.

19. The non-transitory computer-readable storage medium of claim 18, wherein the instructions, when executed by the one or more processors, further cause the computer to determine whether the bowel detected in the data corresponding to the intestinal ultrasound comprises a disease.

20. The non-transitory computer-readable storage medium of claim 18, wherein the instructions, when executed by the one or more processors, further cause the computer to provide an indication to a user of whether the bowel is detected in the data corresponding to the intestinal ultrasound.
